(19) United States
(12) Patent Application Publication
(10) Pub. No.: US 2014/0276605 A1
(43) Pub. Date: Sep. 18, 2014

(54) CATHETER FOR VASCULAR INTERVENTIONS

(71) Applicants: Furqan Tejani, Yonkers, NY (US); Tak Kwan, Staten Island, NY (US)

(72) Inventors: Furqan Tejani, Yonkers, NY (US); Tak Kwan, Staten Island, NY (US)

(21) Appl. No.: 13/840,778

(22) Filed: Mar. 15, 2013

(51) Int. Cl. A61M 25/01; A61M 25/00

(52) U.S. Cl. CPC A61M 25/0152; A61M 25/008
USPC 604/508; 604/523

(57) ABSTRACT

Embodiments provide adjustable vascular catheter systems suitable for use in radial access procedures. In embodiments, the adjustable catheter includes an elongated tubular member having a wall defining a proximal end, a distal end, and a main lumen extending therebetween; a first micro-lumen having a proximal end, a distal end, and a hollow lumen extending therebetween, wherein the first micro-lumen extends substantially outwardly from the catheter wall and runs in parallel with the main lumen; and a first shape wire adapted to pass within the first micro-lumen, wherein the first shape wire has a shape memory function sufficient to deflect a deflectable distal portion of the elongated tubular member. In various embodiments, the shape of the deflected distal portion may allow a user to navigate the catheter past one or more tortuosities and reach normally positioned and anomalous positioned vascular ostia. Some embodiments include second and third shape wires, each having a different shape memory function.

### TECHNICAL FIELD

Embodiments relate to methods and devices for small-vessel access for vascular and cardiac procedures such as diagnostics and interventions, and particularly to methods and devices for radial, brachial, and/or axillary access to the vasculature.

### BACKGROUND

Radial artery access for percutaneous vascular and cardiac interventions and diagnostics has been shown to reduce complications when compared to the standard femoral artery approach. For example, interventions accomplished via the radial artery carry a lower risk of bleeding complications and a higher rate of early ambulation. However, such an approach is complicated and requires a number of steps in order to traverse multiple vascular tortuosities in order to carry out the interventions or diagnostics.

Radial artery access often entails the use of two or more catheters having different shapes adapted for navigating different features of the vasculature. For example, a single procedure might involve multiple Judkins catheters, with each catheter being swapped out as needed during the procedure. These catheter changes increase the length and cost of the procedure, and may involve increased trauma for the patient.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1A-1C illustrate the distal portions of three examples of prior art catheters manufactured by Terumo Cardiovascular, Ann Arbor, Mich., that are typically used for radial artery access to the right and left coronary arteries: a Jacky™ catheter (FIG. 1A), a Tiger™ catheter (FIG. 1B), and a multipurpose catheter (FIG. 1C);

FIG. 2 illustrates an embodiment of an adjustable catheter;

FIG. 3 illustrates a partial cutaway view of the embodiment of FIG. 2, showing three shape wires in a micro-lumen adjacent to the main lumen of the catheter;

FIG. 4 illustrates a close-up view of the embodiment shown in FIGS. 2 and 3;

FIG. 5 illustrates a cross-sectional view of the embodiment shown in FIGS. 2-4;

FIG. 6A illustrates an embodiment of an adjustable catheter with the neutral shape wire advanced and the deflectable distal portion of the catheter adjusted to a neutral position, suitable for performing a left ventricular angiogram;

FIG. 6B illustrates the embodiment shown in FIG. 6A, now with the RCA shape wire advanced and the deflectable distal portion of the catheter adjusted to have a shape suitable for accessing the right coronary artery or right main coronary artery;

FIG. 6C illustrates the embodiment shown in FIG. 6A, now with the LCA shape wire advanced and the deflectable distal portions of the catheter adjusted to have a shape suitable for accessing the left coronary artery or left main coronary artery;

FIGS. 7A-7D illustrate four examples of four alternate shapes the deflectable distal portion of the adjustable catheter may be configured to adopt; and

FIG. 8 illustrates an alternate embodiment of the adjustable catheter; all in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide catheters having shape-shifting capabilities that may enable the use of a single catheter in place of the multiple catheters typically required for diagnostics and interventions using radial artery access. Because a physician may change the shape of the distal portion of the device in real time to conform to the patient's anatomy, he or she may enable the catheter to better conform to the presenting anatomy, and may prevent the need for the exchange of guide catheters mid-procedure in order to find one that is shaped appropriately for the anatomy. Such exchanges involve additional product costs for each additional catheter, as well as additional procedure time and additional risk to the patient as the physician manipulates more devices within the patient's vasculature.

In various embodiments, a catheter in accordance with various embodiments may include an elongated tubular member having a proximal end and a distal end with a main lumen running therebetween, and a micro-lumen running in parallel to the main lumen. In various embodiments, the micro-lumen may be sized and shaped to accommodate at least one shape wire, such as a Nitinol™ shape wire. In various embodiments, the tubular member may include a deflectable distal portion at its distal end, and a stiffer main body portion extending from the deflectable distal portion to the proximal end.

In various embodiments, at least one shape wire may extend through the micro-lumen from the proximal end to the distal end of the adjustable catheter, and in some embodiments, the distal end of the micro lumen may include a closed, blind end near the open distal end of the main lumen. In various embodiments, the shape wire may have sufficient stiffness to deflect the deflectable distal portion of the catheter when advanced distally in the micro-lumen, but it also may lack the stiffness necessary to deflect the stiffer main body of the catheter. In some embodiments, the shape wire may have a shape memory, which may cause it to adopt a predetermined shape when advanced into the deflectable distal portion of the catheter, and thus may cause the deflectable distal portion to adopt the predetermined shape when advanced distally within the micro-lumen.

Thus, in use, the deflectable distal portion of the catheter may have a first shape before the shape wire is advanced towards the distal end of the micro-lumen, and advancing the shape wire towards the distal tip of the catheter within the micro-lumen may allow the shape wire to change the shape of the deflectable distal portion, causing the deflectable distal portion to adopt a second shape. In various embodiments, when the shape wire is again withdrawn from the deflectable distal portion, the deflectable distal end of the catheter may assume the first shape once again. Thus, in various embodiments, advancing and/or withdrawing the shape wire within the micro-lumen may allow a user to change the shape of the deflectable distal portion of the catheter while the catheter is in use.

Some embodiments of the adjustable catheter may include a plurality of shape wires, each producing a different shape when advanced towards the deflectable distal portion, whether singly or in combination with one or more other shape wires. In some embodiments, the plurality of shape wires may all pass within a single micro-lumen, whereas in other embodiments, separate micro-lumens may be provided to accommodate each shape wire. In various embodiments, the adjustable catheter may include one, two, three, or even more shape wires.

Other embodiments provide methods for radial, brachial, or femoral access to various anatomical regions to treat patients having any of several cardiovascular conditions using the adjustable catheters disclosed herein.

FIGS. 1A-1C illustrate the distal portions of three examples of prior art catheters manufactured by Terumo Cardiovascular, Ann Arbor, Mich., that are typically used for radial artery access to the right and left coronary arteries: a Jacky™ catheter (FIG. 1A), a Tiger™ catheter (FIG. 1B), and a multipurpose catheter (FIG. 1C). In various embodiments, during a catheterization procedure, all three of these catheter shapes may be needed to navigate different parts of the patient's anatomy, and one catheter may be removed and replaced with another during the procedure, sometimes several times sequentially. Although the illustrated examples are all manufactured by Terumo Cardiovascular, other manufacturers make other radial access catheters having distal portions with other curved profiles. Although the three illustrated prior art catheters were selected as examples for illustration in the present disclosure, one of skill in the art will appreciate that the adjustable catheters of the present disclosure may be configured to adopt any distal portion profile, be it straight, angled, or curved, or having a combination of straight, angled, and/or curved portions.

FIG. 2 illustrates an embodiment of an adjustable catheter 100, wherein catheter 100 includes a proximal end 102, a distal end 104, a main lumen 110 running therebetween, and a micro-lumen 112 running parallel to main lumen 110. Micro-lumen 112 may be secured to main lumen 110 in any suitable fashion, such as by heat-bonding, co-extrusion, using glue, or any other bonding or extrusion process known to those of skill in the art, in accordance with various embodiments. In various embodiments, micro-lumen 112 may be sized and shaped to accommodate at least one shape wire (not shown), each having a proximal end and a distal end (not shown), and in some embodiments, the shape wires each may include a grip portion 114a, 114b, 114c at its respective proximal end. In some embodiments, grip portion 114a, 114b, 114c may be sized and shaped to allow a physician to easily manipulate the shape wires, particularly by selectively advancing or withdrawing each shape wire within micro-lumen 112 as desired. Apertures 116, or which in the illustrated embodiment are longitudinal slots, may be provided at or near the proximal ends of the shape wires, for example to facilitate advancement or withdrawal of the shape wires within micro-lumen 112, in accordance with various embodiments.

In various embodiments, catheter 100 may be made largely from PTFE or any other thermally-resistant, lubricious and/or hydrophobic material suitable for use within the human body. In some embodiments, catheter 100 may be reinforced, for example with metal braids or helices, such as stainless steel or Nitinol™ braids or coils. Without being bound by theory, such reinforcing materials may help prevent kinking and also may facilitate torque transfer along the catheter. In particular embodiments, one or more radiopaque marker bands or other radiopaque features may be provided to aid in visualization of catheter 100 during use.

Some embodiments of catheter 100 also may include a deflectable distal portion 108 and a stiffer main body portion 106. In some embodiments, deflectable distal portion 108 may have one end that corresponds to distal end 104 and a second, proximal end that merges into main body portion 106 and/or from which main body portion 106 extends. Catheter 100 also may include an intermediate portion (not shown) that is positioned between stiffer main body portion 106 and distal portion 108, such that the transition between deflectable and stiffer portions of catheter 100 may be more gradual. In some embodiments, deflectable distal portion 112 and/or distal tip 104 may include or be made of a soft polymer, such as a polyether-based polyamide.

In some embodiments, the deflectability of deflectable distal portion 108 and stiffer main body portion 106 may be modulated by increasing (e.g., in stiffer main body portion 106) or decreasing (e.g., in deflectable distal portion 108) the thickness of the catheter wall. In other embodiments, the deflectability of the respective proximal and distal portions may be modulated by varying the catheter wall material, for example by including a catheter wall material having a higher or lower elastic modulus, density, or durometer. In other embodiments, the deflectability of the respective proximal and distal portions may be modulated by adjusting the amount or placement of reinforcing material, such as Nitinol™ helices or braids in respective portions of the catheter wall.

In various embodiments, deflectable distal portion 112 may be sufficiently deformable and/or compliant such that advancement of one or more shape wires to the distal tip of micro-lumen 112 may allow the shape wire to deform or deflect catheter 100, such that distal tip 104 may be raised, lowered, curved, straightened, flared outward, or a combination thereof, in response to shape-shifting pressure from the shape wire within micro-lumen 112. In the illustrated embodiment, each grip portion 114a, 114b, 114c may be selectively advanced or withdrawn by the physician, thereby selecting which shape wire(s) to advance distally or withdraw proximally. The illustrated embodiment shows deflectable distal portion 108 in a neutral configuration, which corresponds to having grip feature 114c (and its corresponding shape wire, not shown) advanced into the distal-most position of micro-lumen 112, and providing shape-shifting forces therein to deflect the deflectable distal portion 108. In some embodiments, deflectable distal portion 108 also may be preconfigured to adopt a particular shape even when no shape wires are advanced within the micro-lumen 112.

FIG. 3 illustrates a partial cutaway view of the adjustable catheter 100 of FIG. 2, showing three shape wires 118 in a micro-lumen 112 adjacent to the main lumen 110 of the catheter 100. In various embodiments, micro-lumen 112 may be sized and shaped to accommodate one, two, or three shape wires 118, or even more. In the illustrated embodiment, all three shape wires 118 are housed within a single micro-lumen 112, although in other embodiments, separate micro-lumens 112 may be provided for each shape wire 118. FIG. 4 illustrates a close-up view of the catheter 100 shown in FIGS. 2 and 3, and shows that each of grip portions 114a, 114b, 114c engages a corresponding shape wire 118a, 118b, 118c.

FIG. 5 illustrates a cross-sectional view of the adjustable catheter 100 shown in FIGS. 2-4, and shows how each grip portion 114a, 114b, 114c couples to a corresponding shape wire 118a, 118b, 118c. Although the illustrated example shows each grip portion 114a, 114b, 114c extending through an aperture 116 to reach each shape wire 118a, 118b, 118c within micro-lumen 112, one of skill in the art will appreciated that other configurations are contemplated, such as grip portions 114a, 114b, 114c coupled to the proximal end of each shape wire 118a, 118b, 118c and extending out of an open proximal end (not shown) of micro-lumen 112. Additionally, in the illustrated embodiment, micro-lumen 112 and main lumen 110 are both shown as being formed from a continuous wall 120 having a substantially uniform thickness. One of skill in the art will appreciate that in other embodiments, the respective walls of the micro-lumen 112 and main lumen 110 may be separate structures, for example separate structures bonded together, and may have different thicknesses and/or may be made from different materials.

FIG. 6A illustrates an embodiment of an adjustable catheter 100 in which the neutral shape wire grip portion 114c (and the corresponding shape wire, not shown) is advanced towards the deflectable distal portion 108, thereby deflecting the deflectable distal portion 108 and the distal tip 104 of the catheter to a neutral position, suitable for performing a left ventricular angiogram. In the illustrated embodiment, advancing the neutral shape wire past the stiffer main body portion 106 of catheter 100 and into the deflectable distal portion 108 of catheter 100 may allow it to affect the shape of the deflectable distal portion 108 and deflect it to the illustrated neutral position. By contrast, the other two shape wire grip portions 114a, 114b are retracted proximally, and their corresponding shape wires (not shown) are retracted out of the deflectable distal portion 108. Thus, the retracted shape wires cannot affect the shape of the deflectable distal portion. Thus retracted, the shape wires do not have the force that would be necessary to deflect stiffer main body portion 106, and thus cannot affect the shape of any portion of catheter 100.

FIG. 6B illustrates the embodiment shown in FIG. 6A, now with the RCA shape wire grip portion 114a (and the corresponding shape wire, not shown) advanced towards the deflectable distal portion 108, thereby deflecting the deflectable distal portion 108 and the distal tip 104 of the catheter to a shape suitable for accessing the right coronary artery or right main coronary artery. In the illustrated embodiment, advancing the RCA shape wire past the stiffer main body portion 106 of catheter 100 and into the deflectable distal portion 108 of catheter 100 may allow it to affect the shape of the deflectable distal portion 108 and deflect it to the illustrated position. By contrast, the other two shape wire grip portions 114b, 114c are retracted proximally, and their corresponding shape wires (not shown) are retracted out of the deflectable distal portion 108. Thus, the retracted shape wires cannot affect the shape of the deflectable distal portion 108. Thus retracted, the shape wires do not have the force that would be necessary to deflect stiffer main body portion 106, and thus cannot affect the shape of any portion of catheter 100.

FIG. 6C illustrates the embodiment shown in FIG. 6A, now with the LCA shape wire grip portion 114b (and the corresponding shape wire, not shown) advanced towards the deflectable distal portion 108, thereby deflecting the deflectable distal portion and the distal tip of the catheter to have a shape suitable for accessing the left coronary artery or left main coronary artery. In the illustrated embodiment, advancing the LCA shape wire past the stiffer main body portion 106 of catheter 100 and into the deflectable distal portion 108 of catheter 100 may allow it to affect the shape of the deflectable distal portion 108 and deflect it to the illustrated position. By contrast, the other two shape wire grip portions 114a, 114c are retracted proximally, and their corresponding shape wires (not shown) are retracted out of the deflectable distal portion 108. Thus, the retracted shape wires cannot affect the shape of the deflectable distal portion 108. Thus retracted, the shape wires do not have the force that would be necessary to deflect stiffer main body portion 106, and thus cannot affect the shape of any portion of catheter 100.

FIGS. 7A-7D illustrate four examples of four alternate shapes the adjustable catheter may be configured to adopt in accordance with various embodiments. Although four exemplary shapes are illustrated, one of skill in the art will appreciate that the disclosed adjustable catheters may be designed to have any combination of curves, straight portions, and/or angles in the deflectable distal portion, depending on the shape of the shape wire inserted into the micro-lumen.

FIG. 8 illustrates an alternate embodiment of the adjustable catheter 200, in which each grip portion 214a, 214b, 214c couples to a corresponding shape wire 218a, 218b, 218c, which passes through a corresponding individual micro-lumen 216a, 216b, 216c. Although a particular grip portion 214 is illustrated, one of skill in the art will appreciate that grip portions 214 may be replaced with label tabs, color coded elements, or the like, for instance to help a physician distinguish multiple shape wires. Additionally, one of skill in the art will appreciate that although grip wires 218 are illustrated as entering separate micro-lumens 216, the adjustable catheter also may be configured such that a single micro-lumen 216 may accommodate more than one shape wire 218 at a time.

As described above, the disclosed adjustable catheters are particularly useful for small vessel access, for example radial and brachial access, for example for coronary, neurological, and peripheral procedures. Providing access from the arm has advantages in enabling patients to be discharged and mobile with only a bandage, and often no closure devices are necessary. In contrast, femoral access has higher costs associated with patient management, as a closure device may be needed, and mobility of the patient may be limited, which may significantly delay patient discharge and thereby may reduce the frequency and the number of patients that may be treated.

In one example of a procedure using radial access for cardiac trans-endocardial delivery or a left ventricular procedure, a wire may be inserted into the radial artery using but not limited to double wall stick technique. A sheath is inserted with a dilator and the sheath provides the hemostatic device access for the catheter advancement. In other embodiments the catheter may be inserted without a sheath. In various embodiments, the adjustable catheter may have an internal diameter of between 4 French and 5.5 French and an outer diameter of between 6 French and 7.5 French, and also may have a lubricious coating on at least part of its distal length. The catheter may be inserted over a guidewire. In these embodiments, contrast dye is infused through the device to assess with obtaining an anatomy roadmap. The adjustable catheter may then be advanced into the aorta.

In various embodiments, the deflectable distal portion of the adjustable catheter may be used to guide the guidewire across the aortic valve, the deflection being controlled by the insertion and/or withdrawal of appropriate shape wires within the micro-lumen, after which the adjustable catheter may be passed into the left ventricular chamber. In various embodiments, the torque transmitting ability of the adjustable catheter and the adjustability of the deflectable distal portion of the catheter may be used to form a conduit that may be pointed towards any wall region within the left ventricle. In various embodiments, this conduit may have an internal diameter (main lumen size) of up to 5.5 French (1.8 mm), which is sufficient for delivering a stent or any other interventional devices.

In another embodiment, the distal portion of the adjustable catheter could be directed into any of the coronary artery Ostia or the coronary bypass grafts in the aorta for access to obtain imaging with contrast to evaluate for Stenosis and also to deliver guidewire and coronary stent or other devices for interventional procedures.

In another embodiment, the adjustable catheter disclosed herein may be used as renal and mesenteric access sheaths. In one example, a 6 French adjustable catheter may be used that has long gentle curve such that the deflection of the deflectable distal portion results in the adjustable catheter having back up support from the distal aorta. Devices generally used for treatment of renal artery disease are the renal double curve catheters. In one example, an adjustable catheter suitable for such use would include a curved region in a shape that is substantially similar to a first curve of a renal double curve catheter (RDC1) and a distal second curve formed by the deflectable distal portion with a length of about 3 cm to about 5 cm, wherein the deflectable distal portion is deflected by advancing a suitable shape wire within the micro-lumen to navigate around a tight radius of curvature of about 1 cm within a plane defined by the shape defining the first of the RDC1 curves.

In another example, the adjustable catheters described herein may be used for carotid access. Careful manipulation is essential when accessing the carotid in order to avoid strokes caused by emboli dislodged in the non-target vessel. In various embodiments, carotid access may be performed from either the femoral artery or a brachial artery in which a 180 degree turn is made to ascend to the carotid. Generally two shapes with proximally located prefixed bends are used (such as the Vitek™ shaped catheter). In one embodiment, an adjustable catheter having a preformed shape may be provided. The preformed shape may be substantially similar to that of the Vitek™ catheter, and the deflectable distal portion may have a length of about 3 cm to about 5 cm. In various embodiments, when deflected by insertion and/or withdrawal of the appropriate shape wires within the micro-lumen, the deflectable distal portion may deflect around a radius of curvature of about 1 cm away from the proximal end of the catheter and within the plane defined by the preformed shape.

In a second embodiment suitable for carotid access, the adjustable catheter may have a preformed curve in a shape substantially similar to that of the HIT^{M} catheter, and the deflectable distal portion of the device may have a length of about 3 cm to about 5 cm, and may be deflected by advancement and/or withdrawal of the appropriate shape wires within the micro-lumen around a tight radius of curvature of about 1 cm towards the proximal end of the catheter.

On the right side of the heart, and adjustable catheter that functions as a guide or introducer system may provide a means to control the key procedures performed in these chambers of the heart, namely gaining access to the coronary sinus, and performing right heart biopsies for assessing cardiac transplant rejection. Here, and adjustable catheter system has value for optimizing position of cardiac bioptomes, which may be recorded in biplanar screen overlays or using an MRI Fusion System such as has been developed for cell therapy, such that each biopsy site is recorded and not repeated. In various embodiments, this may enable the biopsies (performed four times per procedure with up to 20 procedures in the first year post heart transplant) to not hit the same spot twice and to avoid scar tissue, which eliminates the value of the biopsies. Such and adjustable catheter-based biopsy system with a means to record locations also has value for biopsies used to obtain tissues for obtaining tissue to culture ex vivo for subsequent cardiac repair and regeneration strategies.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An adjustable catheter system comprising:
an elongated tubular member having a wall defining a proximal end, a distal end, and a main lumen extending therebetween;
a first micro-lumen having a proximal end, a distal end, and a hollow lumen extending therebetween, wherein the first micro-lumen extends substantially outwardly from the catheter wall and runs in parallel with the main lumen; and
a first shape wire adapted to pass within the first micro-lumen, wherein the first shape wire has a shape memory function sufficient to deflect a deflectable distal portion of the elongated tubular member.

2. The adjustable catheter system of claim 1, wherein the elongated tubular member comprises a proximal portion extending proximally from the deflectable distal portion.

3. The adjustable catheter system of claim 1, wherein the deflectable distal portion is more flexible than the proximal portion.

4. The adjustable catheter system of claim 3, wherein the proximal portion has a higher density than the deflectable distal portion.

5. The adjustable catheter system of claim 3, wherein the proximal portion has a higher durometer than the deflectable distal portion.

6. The adjustable catheter system of claim 3, wherein the proximal portion has a higher elastic modulus than the deflectable distal portion.

7. The adjustable catheter system of claim 3, wherein a thickness dimension of the wall is greater in the proximal portion than the thickness dimension of the wall in the deflectable distal portion.

8. The adjustable catheter system of claim 1, wherein the micro-lumen comprises a closed distal end.

9. The adjustable catheter system of claim 1, wherein the system further comprises:
a second micro-lumen having a proximal end, a distal end, and a hollow lumen extending therebetween, wherein the second micro-lumen extends substantially outwardly from the catheter wall and runs in parallel with the main lumen; and
a second shape wire adapted to pass within the second micro-lumen, wherein the second shape wire has a shape memory function sufficient to deflect the deflectable distal portion of the elongated tubular member.

10. The adjustable catheter system of claim 9, wherein the system further comprises:
a third micro-lumen having a proximal end, a distal end, and a hollow lumen extending therebetween, wherein the third micro-lumen extends substantially outwardly from the catheter wall and runs in parallel with the main lumen; and
a third shape wire adapted to pass within the third micro-lumen, wherein the third shape wire has a shape memory function sufficient to deflect the deflectable distal portion of the elongated tubular member.

11. The adjustable catheter system of claim 10, wherein the first, second, and third shape wires each have a different shape memory function.

12. The adjustable catheter system of claim 1, wherein the system further comprises:
a second shape wire adapted to pass within the first micro-lumen, wherein the second shape wire has a shape memory function sufficient to deflect the deflectable distal portion of the elongated tubular member.

13. The adjustable catheter system of claim 1, wherein the system further comprises:
a third shape wire adapted to pass within the first micro-lumen, wherein the third shape wire has a shape memory function sufficient to deflect the deflectable distal portion of the elongated tubular member.

14. The adjustable catheter system of claim 13, wherein the system further comprises:
first, second, and third grip portions coupled respectively to the first, second, and third shape wires, wherein the first, second, and third grip portions are visually distinguishable from one another.

15. The adjustable catheter system of claim 13, wherein the system further comprises:
first, second, and third grip portions coupled respectively to the first, second, and third shape wires, wherein the first, second, and third grip portions are distinguishable from one another by touch.

16. The adjustable catheter system of claim 13, wherein the first, second, and third shape wires each have a different shape memory.

17. The adjustable catheter system of claim 13, wherein the first, second, and third shape wires each comprise Nitinol™.

18. A method of using an adjustable vascular catheter, the method comprising:
inserting the adjustable catheter into the vasculature of a subject, wherein the adjustable catheter comprises a proximal portion and a deflectable distal portion, the deflectable distal portion having a first shape;
advancing a first shape wire distally within a micro-lumen coupled to and running parallel to a main lumen of the adjustable catheter, the first shape wire having a shape memory function;
deflecting a deflectable distal portion of the adjustable catheter with the first shape wire to cause the deflectable distal portion to assume a second shape; and
advancing the adjustable catheter within the vasculature past a tortuosity.

19. The method of claim 18, wherein the method further comprises withdrawing the first shape wire within the micro-lumen to allow the deflectable distal portion to re-assume the first shape.

20. The method of claim 19, wherein the method further comprises;
advancing a second shape wire distally within the micro-lumen, the second shape wire having a shape memory function; and
deflecting the deflectable distal portion with the second shape wire to cause the deflectable distal portion to assume a third shape.
